(19) United States
(12) Patent Application Publication
(10) Pub. No.: US 2023/0372625 A1
(43) Pub. Date: Nov. 23, 2023

(54) DEVICE FOR INTESTINAL DRUG DELIVERY

(71) Applicant: Novo Nordisk A/S, Bagsvaerd (DK)

(72) Inventor: Morten Revsgaard Frederiksen, Koebenhavn K (DK)

(21) Appl. No.: 18/027,528

(22) PCT Filed: Oct. 1, 2021
(86) PCT No.: PCT/EP2021/077061

§ 371 Date: Mar. 21, 2023

(30) Foreign Application Priority Data
Oct. 2, 2020 (EP) 20199760.8

(51) Int. Cl. A61M 5/30

(52) U.S. Cl. CPC A61M 5/3007; A61M 2210/106

(57) ABSTRACT

An ingestible jet injection device for introducing a fluid drug formulation into a gastrointestinal wall portion of a living mammal subject comprises an adaptive nozzle, a drug reservoir in flow communication with the nozzle, and a drive arrangement for pressurizing the fluid drug, the drive arrangement being actuatable from an initial state to a released state. The nozzle comprises an elastomeric nozzle member with an axially fixed, radially distensible outlet channel, the channel being fully collapsed when the drive arrangement is in the initial state thereby providing a sealed outlet from the reservoir. The channel opens when the drive arrangement is actuated and the pressure in the reservoir exceeds a given opening pressure level thereby creating a collimated jet stream of fluid drug adapted to penetrate a gastro-intestinal mucosal surface, the cross-sectional area of the channel adapting in response to variation in reservoir pressure.

The present invention relates to drug delivery devices and systems adapted for delivery of a drug formulation to the intestinal mucosal layer.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise since protein-based drugs will be degraded and digested rather than absorbed when ingested.

To provide a working solution for delivering insulin into the bloodstream through oral intake, the drug has to be delivered into the wall of the gastrointestinal tract (GI wall). This presents several challenges among which are: (1) The drug has to be protected from degradation or digestion by the acid in the stomach. (2) The drug has to be released while being in the lower gastrointestinal tract, i.e. after the stomach, which limits the window of opportunity for drug release. (3) The drug has to be delivered at the GI wall to limit the time exposed to the degrading environment of the fluids in the lower gastrointestinal tract. If not released at the wall, the drug may be degraded during its travel from point of release to the wall or may pass through the lower gastrointestinal tract without being absorbed, unless being protected against the decomposing fluids. (4) The drug has to pass the mucosal layer of the GI wall and penetrate into the richly vascularized submucosal layer the GI wall, which is challenging for larger molecules such as proteins.

For this purpose ingestible delivery devices have been proposed which rather than passive chemical absorption provides that a drug substance is introduced into the GI wall. Such a device comprises an amount of a drug formulation, an onboard actuation and driving arrangement, as well as means for self-triggering the driving arrangement. The drug may in the form of a solid pellet which is “shot” into the GI wall, or it may be in fluid form injected through a short needle introduced into the GI wall or via needleless jetting (jet injection) through a nozzle. In general, to properly deliver the drug into the GI wall the drug outlet from the ingestible device would have to be arranged in proximity to or in contact with the mucosal lining of the GI wall.

An ingestible jetting device may be driven by e.g. a compressed gas or a spring. During storage and until released the nozzle may be sealed by e.g. a dissolvable coating or a plug.

WO 2020/106704 discloses an ingestible jet injection device for introducing a drug substance into an intestinal wall portion via one or more nozzle openings, each nozzle having a desired size and shape appropriate for the desired type of delivery of a dispensable substance from the ingestible device. The ingestible device comprises a restraining mechanism having a first state in which it is configured to prevent the dispensable substance from exiting the ingestible device via the nozzle(s), and a second state in which it is configured so that it does not prevent the dispensable substance from exiting the ingestible device via the nozzle(s).

US 2016/0184525 discloses a supply system for pulsed jet delivery of a cell suspension using a first fluid at a first penetrating pressure and a second cell-containing fluid suspension at a second lower pressure. To prevent dripping between delivery of the first and second fluid a flexible nozzle is utilized which ensures that the nozzle is closed between the pulses. The flexible nozzle is configured to allow a certain axial expansion without opening, this allowing a pre-dosed amount of fluid to be received in a distal reservoir upstream of the valve before pulsed jet injection takes place. The elastomeric nozzle has a circumferential lip-formed configuration around the nozzle opening.

US 2009/0001196 discloses a droplet jet system for oral care, the system comprising an initially open flexible nozzle member which distorts forwardly during fluid flow to reduce the diameter and which can expand when clocked during operation, the wider nozzle diameter allowing the clogging substance to be flushed out of the nozzle opening.

Having regard to the above, it is an object of the present invention to provide an ingestible drug delivery assembly or device which efficiently, reliably and cost-effectively allows a drug formulation to be delivered in the gastrointestinal tract and absorbed into the bloodstream.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention an ingestible jet injection device for introducing a drug substance into an intestinal wall portion of a living mammal subject is provided. The device comprises a housing, an adaptive nozzle, a reservoir in flow communication with the nozzle and containing a fluid drug, and a drive arrangement for pressurizing the fluid drug, the drive arrangement being actuatable from an initial state to a released state. The nozzle comprises an elastomeric nozzle member with an axially fixed, radially distensible outlet channel, the channel being fully collapsed when the drive arrangement is in the initial state thereby providing a sealed outlet from the reservoir. The channel is adapted to open when the drive arrangement is actuated and the pressure in the reservoir exceeds a given opening pressure level thereby creating a collimated jet stream of fluid drug adapted to penetrate a gastro-intestinal mucosal surface, wherein the cross-sectional area of the channel adapts in response to variation in reservoir pressure.

The term “axially fixed” indicates that the outlet channel (i.e. the part of the nozzle member defining the channel) is not designed to move axially and for all practical purposes do not move axially during operation. However, an elastomeric nozzle member may never-the-less move axially to a minimal extent, e.g. less than 0.5 mm, less than 0.3 or less than 0.1 mm during operation.

By the above arrangement the initially collapsed channel provides a sealing barrier between the exterior and the reservoir interior, this allowing a simple design not relying on e.g. mechanical restraining mechanisms or dissolvable nozzle coatings which may interfere with nozzle operation. Such a coating may or may not be included. Further, the arrangement would allow the nozzle to be dynamically tailored to the characteristics of the created jet to thereby optimize the effectiveness of the ingestible drug delivery device. The channel when open may have a generally circular cross section and a length of e.g. more than 0.3 mm, more than 0.5 mm or more than 1 mm.

In an exemplary embodiment the nozzle is in the form of a nozzle assembly comprising an inner part in the form of the elastomeric nozzle member, and an outer part having a fixed-size opening aligned with the channel thereby providing a nozzle maximum aperture.

The drive arrangement may comprise e.g. a compressed helical spring, a compressed gas or gas generating means as an energy source. The drive arrangement may be provided with a release mechanism comprising an initially blocking release member formed from a material dissolvable when exposed to gastrointestinal fluid. In the context of the present application the term “dissolve” also covers processes which may be characterized as erodes and/or degrades.

As used herein, the term “drug” is meant to encompass any drug formulation capable of being passed through a delivery and into the specified target site. The drug may be a single drug compound or a premixed or co-formulated multiple drug compounds drug agent from a single reservoir. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid, powder or liquid form. Specifically, the drug man be an insulin or an GLP-1 containing drugs, this including analogues thereof as well as combinations with one or more other drugs.

### BRIEF DESCRIPTION OF THE DRAWINGS

In the following embodiments of the invention will be described with reference to the drawings, wherein

FIG. 1 shows an exemplary embodiment of an ingestible drug delivery device, and

FIGS. 2A-2D show different states of operation for the device shown in FIG. 1.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

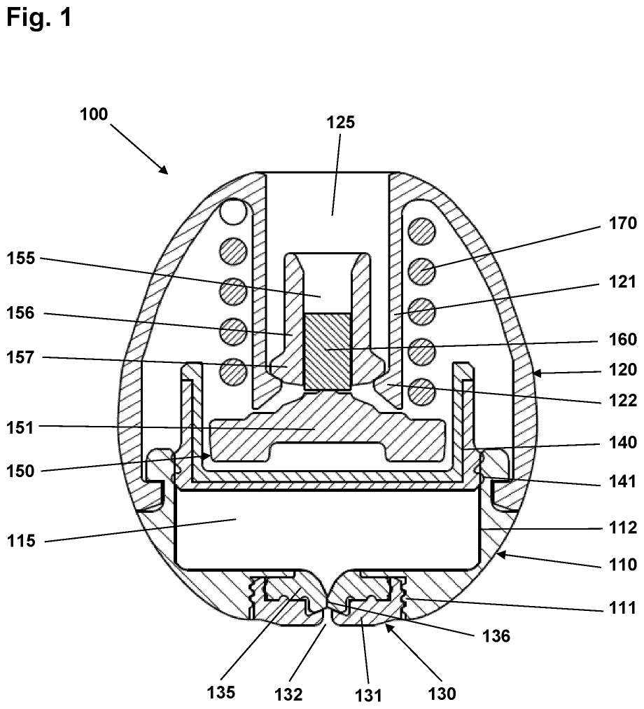

With reference to FIG. 1 a conceptual embodiment of a drug delivery device in accordance with an aspect of the invention will be described, the embodiment being designed to be ingested and release in the gastrointestinal tract (GI) against the GI wall. When designed to release in the stomach it is important that the device is designed to provide the desired positioning and orientation of the device in the gastrointestinal tract (GI) with the nozzle outlet in contact with or in close proximity to the stomach wall. For example, in a self-orienting system, a combination of geometry and mass distribution result in a system which is solely stable in an orientation that aligns the orifice with the tissue. For any free body, if the centre of gravity is not directly above the body's point or line of contact with the ground, a net torque on the body will result. Designed properly, a self-orienting jetting device leverages this torque to roll in the direction of the orifice. When the device centre of gravity is directly above the orifice, there will be no torque on the device, resulting in a stable orientation. When designed to release in the much narrower lumen of the small intestine, the nozzle outlet will in most cases by itself be in contact with or in close proximity to a portion of the intestinal wall. Alternatively, the use of e.g. a balloon structure for nozzle alignment in the small intestine has been proposed.

Assessing jet performance for a given ingestible jetting device it has been found that jet energy and peak power are relevant parameters to describe a given device' ability to effectively deliver an amount of fluid drug to the submucosal space of an intestinal organ.

Assessing jets in terms of energy and peak power take into account all factors affecting depot formation such as jet diameter and velocity of jet. Moreover, makes it easier to compare with devices using other energy sources such as pressurised gas and effervescent reaction.

Power is defined by the following equations:

$${P}^{2} = \frac{{T}^{3}}{4 ρ A} \qquad ( eq . 1 )$$

$$T = ρ {AV}^{2} \qquad ( eq . 2 )$$

P=Power [W], T=Thrust/Force [N], Rho=density [kg/m³], A=area [m²], V=velocity [m/s]

Thrust can be measured using a force transducer (e.g. a Kistler 9215A) where the jetting device is placed and released against the force transducer. Optimal depots in the intestine (highest efficiency) were obtained at jet power of 6.6 W for orifice diameter of 250 μm, density of water and measured force of 0.20N.

The range of peak powers at which depots in the intestine were formed ranges between 5 W and 7 W before perforation is observed. As for stomach tissue, the optimal power to form depot with high efficiency is approximately 21.4 W. However, depots start forming from 15 W and 26.34 W before perforations are observed. Based on equation 2 it is possible to calculate theoretical performance based on the different parameters. A detailed disclosure and discussion of parameters relevant for intestinal jet injection can be found in e.g. WO 2020/106704 which is hereby incorporated by reference.

More specifically, FIG. 1 shows an ingestible jetting device 100 having a generally egg-formed outer shape with the nozzle exit arranged on the distally-facing “base” of the egg. The device comprises a lower housing part 110, a thereto attached upper housing part 120 which in combination provides the outer shape of the device as well as an interior space, a nozzle assembly 130 arranged in the lower part, a piston 140, a combined driver and release member 150, a release plug 160 and a helical drive spring 170.

The lower part 110 comprises a distal bore 111 adapted to receive the nozzle assembly 130 and a proximal reservoir bore 112 adapted to receive the piston 140 in axially sliding engagement to thereby form a variable volume drug reservoir 115 in flow communication with the nozzle. The nozzle assembly comprises an outer rigid part 131 with a distal fixed diameter nozzle opening 132, as well as an elastomeric inner nozzle member 135, e.g. made from high density chlorobutyl, having a variable diameter nozzle outlet channel 136 which in an initial unpressurized state is fully collapsed and thus closed providing a seal between the exterior and the reservoir interior. In this way the provision of an additional sealing component, e.g. a coating or a penetratable membrane, can be dispensed with, this improving reliability and reducing costs. As pressure rises in the reservoir the channel will eventually open with a diameter varying with the pressure, i.e. an adaptive nozzle is provided. In case the channel expands to a diameter larger than the fixed diameter nozzle opening 132 the latter will serve as a maximum nozzle diameter for the nozzle assembly 130. The elastomeric nozzle member is arranged axially fixed such that the channel can only expand radially.

The upper part 120 comprises an axially arranged tubular portion 121 with a proximal outer opening 125 and a distal inner opening, the latter comprising an inner circumferential locking flange 122, and being arranged to receive the release portion of the combined driver and release member 150.

The piston 140 has a generally cup-shaped configuration with a distally-facing outer piston surface and a proximally-extending skirt portion with a distally arranged pair of circumferential elastomeric ridges 141 adapted to be in sliding and sealing engagement with the circumferential wall of the reservoir bore. In the shown embodiment the piston comprises an inner rigid part and a thereto bonded outer elastomeric part. The proximally facing inner piston surface is adapted to engage the driver portion of the combined driver and release member 150.

The combined driver and release member 150 comprises a disc-shaped distal driver portion 151 and a proximally extending tube-formed release portion 155 arranged inside the upper part tubular portion 121. The release portion comprises a pair of opposed axially extending flexible locking arms 156 having distal free locking ends 157 radially moveable between an expanded state in which the locking ends 157 engages the proximally facing surface of the circumferential locking flange 122, and a retracted state in which the locking ends 157 have disengage the locking flange 122 thereby allowing the combined driver and release member 150 to move distally by the drive spring 170.

The flexible locking arms 156 are held in their initial expanded state by a plug-formed release member 160 formed from a dissolvable material and arranged inside the tubular release portion 155 and thus in communication with the exterior through the proximal outer opening 125. For a device that targets e.g. the stomach, triggering should occur between five and twenty minutes after ingestion. The exemplary release “sugar-plug” member is composed of an isomalt which passively degrades in humid environments. When the device is ingested, the sugar-plug begins to dissolve. Eventually, it becomes small enough so that the outwardly biased flexible locking arms 156 are allowed to move inwardly and thus free of the locking flange 122.

Alternatively, the plug may be formed from materials generally termed an “enteric coatings” as they have been designed to ensure that a given coated object will pass the stomach and subsequently enter the intestine. Such coatings are generally known. An enteric coating material may be any suitable coating material that allows the given object to be released in the intestine. In some cases, an enteric coating material may dissolve preferentially in the small intestine as compared to the stomach. In other embodiments, the enteric coating material may hydrolyse preferentially in the small intestine as compared to the stomach. Non-limiting examples of materials used as enteric coatings include methyl acrylate-methacrylic acid copolymers, cellulose acetate succinate, hydroxy propyl methyl cellulose phthalate, hydroxy propyl methyl cellulose acetate succinate (i.e., hypromellose acetate succinate), polyvinyl acetate phthalate (PVAP), methyl methacrylate-methacrylic acid copolymers, and sodium alginate, and stearic acid. Additional examples are disclosed in e.g. US 2018/0193621 hereby incorporated by reference. The enteric coating material may be composed to be soluble at a given pH or within a given pH range, e.g. at a pH greater than 5.5, at a pH greater than 6.5, within a range of about 5.6 to 6 or within a range of about 5.6 to 6.5 or 7. The dissolution time at an intestinal pH may be controlled or adjusted by the composition of the enteric coating material as well as the configuration of the plug, e.g. the dissolution time at an intestinal pH may be controlled or adjusted by the thickness of the plug.

The axially compressed helical drive spring 170 is arranged around the upper part tubular portion 121 with its proximal portion engaging the upper housing part 120 and the distal portion engaging the proximal surface of the disc-shaped driver portion 151 thereby exerting a driving force on the piston and thus the fluid in the reservoir. The drive spring is designed to release only a given portion of the stored energy, e.g. 50% when it expands from its initial state and to its fully expanded state when the piston has been moved to its distal-most position with essentially all of the fluid drug formulation having been expelled through the nozzle 130.

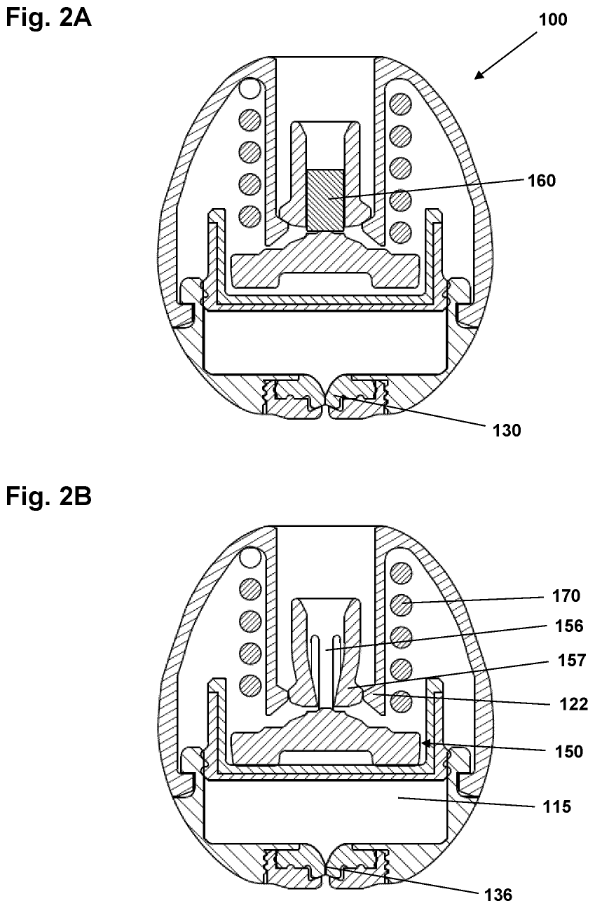

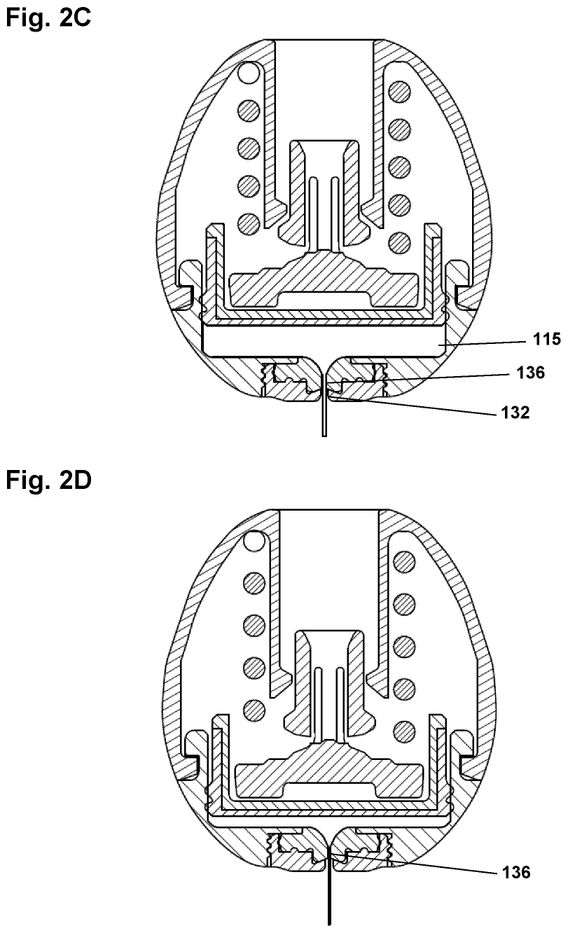

With reference to FIGS. 2A-2D operation of the jetting device will be described.

FIG. 2A shows the jetting device 100 prior to use with the release plug 160 in its initial undissolved configuration. The adaptive nozzle 130 is fully closed due to the elastic property of the material and no system pressure. In this state it works as a primary packaging drug barrier.

The impact of ram effects on the jetting efficiency may increase the effective peak power and thereby penetration. The ram or hammer effect is achieved by leaving a gap (as shown in FIG. 2A) between the driving member and the piston which then drives the liquid formulation into a “burst jet”.

In FIG. 2B the device is activated as the locking arms ends 157 have disengaged the locking flange 122, this allowing the combined driver and release member 150 to move distally driven by the drive spring 170 to engage and drive the piston, however, initial pressure in the reservoir 115 is low due to static and dynamic friction in plunger/reservoir interface. The adaptive nozzle is creating an initial narrow collimated jet stream (not shown) as the channel 132 opens. Although the initial velocity of the jet stream may be high, due to the small diameter of the jet the generated effect (in Watts W) of the jet is relatively low for which reason it may not be able to penetrate a given mucosal surface in the GI tract. However, due to the initial narrow diameter of the jet, the amount of lost, i.e. non-injected, drug formulation is relatively low.

In the shown state the release plug 160 is shown as fully dissolved, however, this is only for illustrative purposes. Under real circumstances the plug will start to dissolve from the outer surface until it has become sufficiently thin and/or porous to no longer being able withstand the radial forces exerted by the flexible locking arms at which point it will brake/disintegrate thus allowing the locking arms to move inwards.

In FIG. 2C the dynamic pressure in the reservoir 115 has risen to a maximum resulting in the nozzle channel 136 expanding to a maximum diameter, however, in the exemplary embodiment the effective nozzle diameter is controlled by the fixed diameter nozzle opening 132. At this point a high effect is delivered by the jet enabling it to penetrate the mucosal layer and inject the fluid drug formulation in the submucosal layer. For example, in vitro experiments have shown that in order to penetrate into the submucosal layer of the small intestine, large intestine and rectum effects in the range 2.9-6.6 W was required whereas to penetrate into the submucosal layer of the stomach and esophagus an effect of >18 W was required. It is to be noted that FIGS. 2C and 2D incorrectly show a gap between the combined driver and release member 150 and the piston.

In FIG. 2D the dynamic pressure in the reservoir has lowered due to the decreasing force exerted by the expanding drive spring. As a result the adaptive nozzle channel 136 has contracted to a narrower diameter which in the shown state is below the fixed diameter nozzle. Thus at this state of delivery both the jet velocity and the jet diameter have diminished resulting in a jet stream delivering a smaller effect, however, at this state it is believed that the initial opening created in the tissue will allow continuous delivery of fluid drug formulation to the submucosal space until the reservoir has emptied.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An ingestible jet injection device for introducing a drug substance into an intestinal wall portion of a living mammal subject, comprising:
a housing,
an adaptive nozzle,
a reservoir in flow communication with the nozzle and containing a fluid drug,
a drive arrangement for pressurizing the fluid drug, the drive arrangement being actuatable from an initial state to a released state,
wherein:
the nozzle comprises an elastomeric nozzle member with an axially fixed, radially distensible outlet channel, the channel being fully collapsed when the drive arrangement is in the initial state thereby providing a sealed outlet from the reservoir,
the channel opens when the drive arrangement is actuated and the pressure in the reservoir exceeds a given opening pressure level thereby creating a collimated jet stream of fluid drug adapted to penetrate a gastro-intestinal mucosal surface, and
the cross-sectional area of the channel adapts in response to variation in reservoir pressure.

2. The jet injection device as in claim 1, wherein the channel when open has a generally circular cross section and a length of at least 0.5 mm.

3. The jet injection device as in claim 1, wherein the nozzle comprises an inner part in the form of the elastomeric nozzle member, and an outer part having a fixed-size opening aligned with the channel thereby providing a nozzle maximum aperture.

4. The jet injection device as in claim 1, wherein the drive arrangement comprises a compressed helical spring.

5. The jet injection device as in claim 1, wherein the drive arrangement comprises a release mechanism comprising an initially blocking release member formed from a material dissolvable when exposed to gastrointestinal fluid.

6. The jet injection device as in claim 2, wherein the channel when open has a generally circular cross section and a length of at least 1.0 mm.

7. The jet injection device as in claim 2, wherein the channel when open has a generally circular cross section and a length of at least 1.5 mm.
